(19) United States
(12) Patent Application Publication
(10) Pub. No.: US 2007/0078280 A1
(43) Pub. Date: Apr. 5, 2007

(54) Process for oxidation of methane to acetic acid

(75) Inventors: Alexis T. Bell, Oakland, CA (US); Mark T. Zerella, Berkeley, CA (US); Argyris Kahros, Woodland Hills, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/242,716

(22) Filed: Oct. 3, 2005

(51) Int. Cl. C07C 51/16

(52) U.S. Cl. 562/549

(57) ABSTRACT

Acetic acid or derivatives such as methyl acetate and acetyl sulfate, are produced from methane by contacting a methane-containing feed with an oxygen-containing gas in the presence of a palladium-containing catalyst and an acid selected from concentrated sulfuric acid and fuming sulfuric acid. The process is carried out using an oxygen partial pressure of from about 30 to about 300 psi and most preferably from about 60 to about 200 psi, a methane partial pressure of from about 100 to about 1000 psi, and most preferably from about 100 to about 400 psi, and a total pressure of oxygen and methane of from about 130 to about 1300 psi, preferably from about 200 to about 600 psi. The process is carried out in the absence of a catalyst promoter.

### BACKGROUND OF THE INVENTION

This invention relates in general to an improved process for the production of acetic acid or a derivative thereof by liquid phase oxidation of methane. In particular the present invention relates to the liquid phase oxidation of methane with an oxygen-containing gas in a strong acid in the presence of a catalyst comprising palladium.

The primary process route used today for production of acetic acid is by catalytic reaction of methanol and carbon monoxide. Such a process, typically termed “carbonylation”, is described in a number of patents and publications. Rhodium, palladium or iridium-containing catalysts have been found especially useful for conducting this reaction. A recent example of a patent on such a process is U.S. Pat. No. 6,472,558 of Key et al., which describes a process for reaction of methanol (and/or a reactive derivative of methanol such as methyl acetate or dimethyl ether) and carbon monoxide in a liquid reaction composition that comprises methyl acetate, methyl iodide, acetic acid, water and a polydentate phosphine oxide, in addition to the iridium catalyst.

Another process route that has been found useful for the production of acetic acid involves the catalytic oxidation of ethane and/or ethylene. Such processes are disclosed, for instance, in U.S. Pat. No. 6,383,977 of Karim et al., U.S. Pat. No. 6,399,816 of Borchert et al. and U.S. Pat. No. 6,706,919 of Obana et al., and U.S. published applications 2003/0158440 of Zeyss et al. and 2004/0031084 of Cook et al. In the processes described in these, a mixed oxide catalyst containing multiple metals such as palladium, molybdenum, vanadium, niobium, antimony, nickel, calcium, and others, is used.

Methane is the lowest molecular weight, and simplest in structure, of the hydrocarbons. Because of the existence of large reserves of methane worldwide it has been considered desirable for some time to develop processes to convert methane to more valuable chemicals. Processes for production of acetic acid from methanol represent an ultimate use of methane, but in current commercial practice, the methane first must be converted to methanol. A process that produces acetic acid directly from methane would be more desirable.

A small amount of work has been conducted so far on the direct conversion of methane to acetic acid, for instance by reaction of methane with carbon dioxide. A process for production of acetic acid by such a reaction was disclosed in the 1924 British patent 226,248 of Dreyfus. The patent describes a process involving gas phase reaction of methane with carbon monoxide and/or carbon dioxide in the presence of a catalyst that preferably contains nickel carbonate. Apparently a mixture of acetic acid, acetaldehyde and possibly acetone is obtained. No data on yields or conversions is contained in this patent.

PCT application WO 96/05163 of Hoechst A. G. describes a gas phase reaction of methane and carbon dioxide to produce acetic acid, using a catalyst containing one or more Group VIA, VIIA and/or VIIIA metals. Selectivities of 70-95% based on methane are asserted; however the application contains no exemplary data.

A number of researchers have investigated production of acetic acid by liquid phase carbonylation of methane with carbon monoxide, due to the favorable thermodynamics of this reaction. See, for instance, Bagno, et al. J. Org. Chem. 1990, 55:4284-4289; Lin, et al., Nature 1994, 368:613-615, Chaepaikin, et al., J. Mol. Catal. A: Chem. 2001, 169:89-98; Nishiguchi, et al., Chem. Lett. 1992, 1141-1142; Nakata, et al. J. Organomet. Chem. 1994, 473:329-334; Kurioka, et al., Chem. Lett. 1995, 244; Fujiwara, et al., Studies in Surface Science and Catalysis 1998, 119:349-353; Taniguchi, et al., Org. Lett. 1999, 1(4):557-559; Asadullah, et al., Tetrahedron Lett. 1999, 40:8867-8871; Nizova et al., Chem. Commun. 1998: 1885; Piao et al., J. Organomet. Chem. 1999, 574:116-120; Yin et al., Appl. Organomet. Chem. 2000, 14:438-442; Reis et al., Angew. Chem. Int. Ed. 2003, 42:821; and Asadullah, et al., Chem. Int. Ed. 2000, 39(14):2475-2478.

More recently, Periana et al., Science, 2003, 301:814 reported an experiment in which acetic acid was directly prepared from methane in the presence of palladium sulfate in concentrated sulfuric acid without the addition of COₓ. Acetic acid and methyl bisulfate were the reaction products. Hydrolysis of these yields acetic acid and an equilibrium mixture of methyl bisulfate and methanol. After 7 h of reaction at 180° C., 82 mM acetic acid and 38 mM methanol were ultimately formed. The reaction as reported was 90% selective to these products, with the only byproduct being CO₂. The overall stoichiometry of the reaction is given as:
2CH₄+4H₂SO₄→CH₃COOH+4SO₂+6H₂O.

However, this process displayed a serious drawback. During the reaction particles of palladium black were formed due to the reduction of Pd(II) to Pd(0). This results in loss of catalytic activity due to loss of soluble palladium. A further drawback to the process is that it consumes sulfuric acid and produces SO₂ as a by-product, which is either wasted or must be recycled back to sulfuric acid via a number of processing steps including reaction with oxygen to give SO₃ which is then reacted with water to give sulfuric acid.

In a more recent publication, Periana et al., Topics in Catalysis 2005 32:169-174, the Periana group further investigated the reaction mechanism involved in this process, ascertaining that both carbon atoms in the acetic acid molecule were derived from methane, more specifically by a combination of methane with methanol that has been produced from methane in the process. However, this work does not involve any improvement on the problem of the loss of Pd catalyst; indeed, comments are made that the system eventually shuts down due to the irreversible formation of metallic palladium.

In another recent publication, Zerella et al., Chem. Commun. 2004, 1948-1949, we described a process for production of acetic acid by reaction of methane with oxygen in the presence of concentrated sulfuric acid. Included in the process was cupric chloride, which served as a promoter of catalytic activity, at least in part by suppressing formation of metallic palladium.

### BRIEF SUMMARY OF THE INVENTION

This invention comprises a process for the production of acetic acid, or derivatives such as methyl acetate and acetyl sulfate, from methane, by contacting a methane-containing feed with an oxygen-containing gas in the presence of a palladium-containing catalyst and an acid selected from concentrated sulfuric acid and fuming sulfuric acid. The process is carried out using an oxygen partial pressure of from about 30 to about 300 psi and most preferably from about 60 to about 200 psi, a methane partial pressure of from about 100 to about 1000 psi, and most preferably from about 100 to about 400 psi, and a total pressure of oxygen and methane of from about 130 to about 1300 psi, preferably from about 200 to about 600 psi. The process is carried out in the absence of a promoter.

In general, the [molar?] ratio of oxygen to methane of from about 1:20 to about 1:1, preferably from about 1:4 to about 1:1 and most preferably from about 1:3 to about 1:1.5.

The process temperature will be from about 100 to about 250° C, preferably from about 150 to about 180° C.

### BRIEF DESCRIPTION OF THE DRAWINGS

FIG. 1 depicts the effect of initial palladium concentration on selectivity to acetic acid.

FIG. 2 depicts the correlation between palladium retention in solution and moles of acetic acid produced per mole of palladium introduced (“TON”)

FIG. 3 depicts the impact of SO₃ concentration on product distribution.

### DETAILED DESCRIPTION OF THE INVENTION

This invention comprises a process for the production of acetic acid, or derivatives such as methyl acetate and acetyl sulfate, from methane, by contacting a methane-containing feed with an oxygen-containing gas in the presence of a palladium-containing catalyst and an acid selected from concentrated sulfuric acid and fuming sulfuric acid. The process is carried out using an oxygen pressure of from about 30 to about 300 psi and most preferably from about 60 to about 200 psi, a methane pressure of from about 100 to about 1000 psi, and most preferably from about 100 to about 400 psi, a total pressure of oxygen and methane of from about 130 to about 1300 psi, preferably from about 200 to about 600 psi. The process is carried out in the absence of a promoter.

In general, the ratio of oxygen to methane is from about 1:20 to about 1:1, preferably from about 1:4 to about 1:1 and most preferably from about 1:3 to about 1:1.5. The process temperature will be from about 100 to about 250° C., preferably from about 150 to about 180° C.

The methane that is introduced into the process may be an essentially pure methane stream, a methane stream that contains various impurities, or a stream that contains methane as one of several components, for example, a methane-containing stream that emanates from a chemical process unit, a natural gas stream, a methane-containing stream produced by a gas generator, a methane-containing off-gas, a biogenic methane stream, and the like. The methane feed to the process may also contain other materials that may be oxidized under the process conditions to form acetic acid, e.g. ethane, propane, butane and higher hydrocarbons. Methanol, dimethyl ether, methyl acetate and methyl bisulfate may also be fed to the process in this stream.

The palladium-containing catalyst may be any palladium-containing material that possesses the necessary catalytic activity for this reaction. Preferred palladium-containing catalysts are palladium salts such as palladium (II) and palladium (IV) sulfates, chlorides, nitrates, acetates, acetylacetonates, amines, oxides, and ligand-modified palladium systems, for example systems containing multiple donor atoms like phosphorus, nitrogen or combinations thereof. Examples of such ligands are phosphines, nitriles, and amines, for example triphenylphosphine, bis(diphenylphosphinopropane), bipyrimidine, bipyridine, acetonitrile, and benzonitrile. The catalyst concentration will range from about 0.001 to about 1.0 M, preferably from about 0.001 to about 0.1M.

The reaction is conducted in the presence of an acid, in which the catalyst is dissolved. Acids suitable for use in this process are concentrated sulfuric acid and fuming sulfuric acid, with the concentration ranging from about 80 to about 130 wt. % H₂SO₄, and most preferably from about 95-100 wt. %. In one embodiment, the process is generally carried out by mixing the methane-containing feed and the oxygen-containing gas, with the acid, with the latter containing the catalyst. The oxygen-containing gas may be introduced together with the methane-containing feed, or separately from it. If the two are introduced separately, their flow may be concurrent or countercurrent. The apparatus or equipment used for the process may be any type of suitable reactor, together with associated equipment, for example, for handling materials flowing into and out of the reactor, recycle streams, waste streams, etc. The reactor may include an agitator to aid in the mixing of the reactants. All parts of the apparatus will be constructed from appropriate materials.

The oxygen-containing gas used in the process may be in the form of molecular oxygen, a commercial mixture of molecular oxygen with an inert gas such as nitrogen, oxygen-enriched air, or air, but is most preferably substantially pure oxygen or a commercial mixture that contains predominantly oxygen.

The process is carried out using an oxygen partial pressure of from about 30 to about 300 psi and most preferably from about 60 to about 200 psi, a methane partial pressure of from about 100 to about 1000 psi, and most preferably from about 100 to about 400 psi, and a total pressure of oxygen and methane of from about 130 to about 1300 psi, preferably from about 200 to about 600 psi. Oxygen and methane partial pressures are expressed in terms of the respective substance since the gases fed to the reaction may contain various amounts of other substances. The process is carried out in the absence of a catalyst promoter.

In general, the ratio of oxygen to methane is from about 1:20 to about 1:1, preferably from about 1:4 to about 1:1 and most preferably from about 1:3 to about 1:1.5.

The primary products of the reaction are acetic acid, methyl bisulfate, methanol, and methanesulfonic acid. Water may be added after the completion of the reaction to hydrolyze any anhydrides and/or esters. However, the presence of water should be avoided in carrying out the process, as water tends to inhibit the desired reactions. Accordingly the process is carried out under anhydrous conditions, or with a water content of no more than about 4% w/w (which may result, for instance, from the use of acid that is less than 100% H₂SO₄). Accordingly, the equipment and feed streams are preferably dried before the process is carried out. The methanol, methyl bisulfate, and methanesulfonic acid can be recycled to the oxidation reactor.

The process may be conducted as a batch or continuous process, as desired, with appropriate equipment. It may be conducted as a single-step process, batch or continuous, with all substances introduced into and removed from a single reactor, or as a two-step process, in which the process is preferably conducted as a continuous process. In a two-step process, for instance, the methane, acid, and catalyst are contacted in the first stage and the reaction products of that stage are passed to a second stage (for example a second reactor or a second portion of a reactor) in which they are contacted with the oxygen-containing gas. Alternately, both steps may be carried out in the same reactor, with methane and oxygen feeds being introduced separately, in an alternating pulsed fashion.

Process temperatures in general may be from about 100 to about 250° C., most preferably from about 150 to about 180° C. Suitable reaction times (for a batch process) range from about 0.5 to about 24 hours, preferably from about 2 to about 4 hours.

As with the Periana et al. process and the process described in Zerella et al., the primary products are acetic acid and methyl bisulfate. As reported in Zerella et al., when the process was run in batch mode for 4 hours under conditions identical to those used in Periana et al., the acetic acid yield was 9 mM. With the inclusion of a cupric salt and oxygen, the acetic acid production increased to 49 mM. However, whereas Periana et al. reported methanol production for a 4 hour batch reaction to an extent about the same as that of the acetic acid, the process of Zerella et al. typically produced substantially higher ratios of acetic acid to methanol.

As described in Zerella et al. the inclusion of copper chloride in the process was considered essential for optimal conversion and selectivity to acetic acid, via maintaining catalyst activity. However, according to the present invention, equivalent or better results can be obtained by careful selection of the reaction conditions, which additionally results in lengthened catalyst life, without the use of a copper chloride promoter.

The results presented in the examples below will clearly demonstrate that the yields of acetic acid and methyl bisulfate are strongly dependent on the partial pressures of CH₄ and O₂, the total oxygen and methane pressure, the concentration of PdSO₄ charged to the reaction solution, and the strength of the H₂SO₄ solution. Of these, however, the initial partial pressures of methane and oxygen, and the total pressure of the two, are the most significant. For a particular charge of PdSO₄, the yield of acetic acid can be maximized by using a high ratio of O₂/CH₄ partial pressures and a high total pressure of CH₄ and O₂. These conditions also increase the selectivity to acetic acid versus methyl bisulfate and the Pd²⁺ retained in solution after reaction. The presence of a small amount of CO (up to about 0.2 atm) in the gas phase enhances the yield of acetic acid, but higher CO partial pressures have the opposite effect. A high yield of acetic acid also preferably requires that the concentration of SO₃ in the solution be ˜18.0 M (96 wt. % H₂SO₄). Sulfur-containing acids such as methanesulfonic acid are also formed from methane. The yield of these products strongly depends on the reaction conditions. Higher reaction temperature, SO₃ concentrations and O₂ partial pressures tend to favor the formation of methanesulfonic acid, methanedisulfonic acid, and sulfoacetic acid relative to acetic acid, and so should be avoided for best results.

The yield of acetic acid, the primary product of methane oxidation, increases with both increasing O₂/CH₄ ratio for a fixed CH₄ partial pressure, and with increasing total reactant pressure for a fixed O₂/CH₄ ratio. The increase in acetic acid yield as a consequence of increasing O₂/CH₄ ratio is accompanied by only a modest loss in selectivity to oxygen-containing organic products, and the increase in total pressure of CH₄ and O₂ at a fixed O₂/CH₄ ratio results in a slight rise in the yield of acetic acid. A significant finding of this work is that the retention of initially dissolved Pd²⁺ can be raised to as high as 96%, by raising the O₂/CH₄ ratio and total feed pressure, and without using a catalyst promoter.

### EXAMPLES AND DISCUSSION OF RESULTS

The following are representative examples of the process of this invention. However, the invention is not limited by them, but only by the claims that follow.

Experimental Procedure

All reactions were carried out in a 50 ml Parr autoclave made of Hastelloy C. To minimize problems with reproducibility due to trace amounts of water retained in crevices of the autoclave head, all parts of the reactor were cleaned and dried thoroughly before each run. The temperature of the reacting mixture was measured with a thermocouple located in a well made of Hastelloy C, which was wrapped with Teflon tape to minimize exposure of the metal to the reaction mixture. The reaction mixture was contained in a glass liner. Unless specified otherwise, all reactions were carried out in 96 wt % H₂SO₄ containing 20 mM PdSO₄ at a temperature of 453 K (180° C.). The initial partial pressures of CH₄ and O₂ were chosen to avoid compositions that would result in an explosive mixture during any part of the reaction. In a typical run, 0.0121 g (20 mM) PdSO₄ (Aldrich) and 3 ml of 96% H₂SO₄ (Aldrich) were added to the liner. After placing the glass liner in the autoclave, it was sealed and purged with N₂. The autoclave was then pressurized at room temperature with 400 psi of CH₄ (99.97%, Praxair) and then with the desired pressure of O₂ (99.993%, Praxair). The reactor was brought to 453 K (180° C.) in ˜15 min and then held at this temperature for 4 h. Upon completion of the reaction, the autoclave was quenched in ice water to <308 K (<35° C.) and then vented. Upon opening the autoclave, the solution was further chilled before adding 3 ml of water.

Liquid-phase reaction products were analyzed using ¹H NMR. D₂O was used in a capillary as the lock reference. A known amount of t-butanol was added to each sample and used as an internal standard for quantification. The liquid-phase products observed were acetic acid (CH₃COOH), methanesulfonic acid (CH₃SO₃H), methanol (CH₃OH), methyl bisulfate (CH₃OSO₃H), sulfoacetic acid (HO₃SCH₂COOH), and methanedisulfonic acid (CH₂(SO₃H)₂). The chemical shifts for these products were as follows: acetic acid, 2.0 to 2.1 ppm; methanesulfonic acid, 2.8 to 2.9 ppm; methanol, 3.3 to 3.4; methyl bisulfate, 3.6 to 3.7; sulfoacetic acid, 4.0 to 4.1 ppm; and methanedisulfonic acid, 4.4 to 4.5.

For analysis of the gas products, a sample of the autoclave head-space gas was taken using a gas-tight syringe upon venting the autoclave. Analysis of the sample was carried out by gas chromatography in order to determine the concentrations of CH₄, CO, and CO₂. The concentrations of liquid phase products were determined from the ¹H NMR analyses, and these measurements were combined with the gas chromatographic analyses of the autoclave head space to determine the amount of COₓ (x=1, 2) produced.

The yield of each liquid-phase product is reported in terms of the concentration of that product observed after a fixed period of reaction. Even though water was added to the reaction mixture prior to ¹H NMR analysis, the reported concentrations are calculated on the basis of the volume of liquid present in the autoclave liner prior to the addition of water. In this pre-hydrolyzed state, no methanol is observed. Only after the addition of water some of the methyl bisulfate is hydrolyzed to methanol; therefore any methanol measured is reported as methyl bisulfate. The carbon selectivity to acetic acid, S_{AcOH}, is given as the moles of carbon in acetic acid divided by the sum of the moles of carbon appearing in all liquid- and gas-phase products. In calculating S_{AcOH}, proper account is taken of the volume of the gas head space in the autoclave and the volume of liquid in the autoclave liner.

The concentration of Pd²⁺ retained in solution after reaction (but before water addition) was determined by UV-visible spectroscopy. The peak located at 440 nm, which has been ascribed to Pd(OSO₃H)₂ [E. S. Rudakov; A. P. Yaroshenko; R. I. Rudakova; V. V. Zamashchikov; Ukr. Khim. Zhur., 1984, 50 (7), 680-684] was used to determine the concentration of Pd²⁺ cations in solution. A solution of known concentration of Pd²⁺ was used as a standard for comparison of peak areas.

The results of these experiments are shown in Tables 1 and 2.

TABLE 1
Effect of O₂ Pressure at 200 psi CH₄. Reaction
Conditions: 3 ml 96% H₂SO₄; 0.0121 g (20 mM)
PdSO₄; 200 psi CH₄; X psi O₂; 180° C.; 4 h.

| | O₂ Pressure (psi) | | | | |
|---|---|---|---|---|---|
| | 0 | 30 | 60 | 75 | 125 |
| CH₃COOH (mM) | 65.7 | 52.1 | 136 | 154 | 181.2 |
| CH₃OSO₃H (mM) | 2.5 | 1.7 | 3.1 | 3.9 | 4.8 |
| CH₃SO₃H (mM) | 3.0 | 7.7 | 15.9 | 25.3 | 29.4 |
| HO₃SCH₂COOH (mM) | 5.9 | 6.2 | 11.6 | 9.9 | 9.3 |
| CH₂(SO₃H)₂ (mM) | 18.2 | 65.1 | 31 | 31 | 32.9 |
| S_{CH3COOH} (%) | 46 | 22 | 41 | 37 | 39 |
| S_{COx} (%) | 44 | 60 | 50 | 54 | 53 |
| Pd Retained (%) | 6.5 | 11 | 13 | 48 | 96 |

TABLE 2
Effect of O₂ Pressure at 400 psi CH₄. Reaction
Conditions: 3 ml 96% H₂SO₄; 0.0121 g (20 mM)
PdSO₄; 400 psi CH₄; X psi O₂; 180° C.; 4 h.

O₂ pressure (psi)

| | 0 | 30 | 100 | 150 |
|---|---|---|---|---|
| CH₃COOH (mM) | 78.5 | 118 | 191 | 284 |
| CH₃OSO₃H (mM) | 3.2 | 5.1 | 5.4 | 6.8 |
| CH₃SO₃H (mM) | 9.2 | 8.2 | 27.0 | 55.5 |
| HO₃SCH₂COOH (mM) | 8.3 | 10.1 | 12.2 | 16.3 |
| CH₂(SO₃H)₂ (mM) | 44.6 | 26.3 | 25.4 | 36.8 |
| S_{CH3COOH} (%) | 51 | 57 | 50 | 44 |
| S_{COx} (%) | 27 | 31 | 41 | 47 |
| Pd Retained (%) | 3.8 | 3.3 | 16 | 40 |

Table 1 shows that for an initial CH₄ partial pressure of 200 psi, the yield of acetic acid rises from 65.7 mM to 181 mM as the initial partial pressure of O₂ increases from 0 to 125 psi. Over the same range of O₂ partial pressures, the yield of methyl bisulfate increases from 2.5 mM to 4.8 mM, whereas the production of methanesulfonic acid increases, from 3.0 to 29.4 mM. The other two sulfur-containing byproducts, sulfoacetic acid and methanedisulfonic acid, remain relatively constant at about 9 and 30 mM, respectively. Notably, the overall selectivity of methane conversion to acetic acid decreases only slightly from 46% to 39%.

The most remarkable effect of O₂ partial pressure, though, is on the retention of initially dissolved Pd in solution at the end of the reaction. Table 1 shows that this figure rises from 6.5% to 96% as the initial O₂ partial pressure increases from 0 to 125 psi. Raising the initial CH₄ pressure to 400 psi increases the yield of acetic acid and to a much lesser degree, the yield of methyl bisulfate, as shown in Table 2. Here again, increasing the initial partial pressure of O₂ increases the yield of acetic acid as well as methanesulfonic acid, increases the yield of methyl bisulfate slightly, decreases the selectivity to acetic acid slightly, and increases significantly the percentage of Pd retained in solution. The yield of sulfoacetic acid and methanedisulfonic acid also increase slightly.

Comparison of the results in Tables 1 and 2 reveals that for the same O₂/CH₄ ratio, operation at higher initial pressures of both reactants results in an increase in the yield of acetic acid that is essentially proportional to the increase in the total initial pressure, and to only a small increase in the yield of methyl bisulfate. The overall selectivity of methane conversion to acetic acid and methyl bisulfate increases slightly for operation at higher pressure, but the retention of the initially dissolved Pd as Pd²⁺ remains essentially the same. It is noted that while reaction with the O₂ and CH₄ pressures of 250 psi and 400 psi would be expected to result in complete retention of Pd²⁺ in solution, these conditions were not used in these laboratory experiments because this reaction mixture lies within the explosive region for a batch reactor.

We also explored the effects of adding CO into the gas phase. At very low pressures (˜0.02 atm), CO has a beneficial effect, boosting the acetic acid yield from 78 mM to 109 mM. However, further increasing the CO pressure inhibited the reaction. Once the partial pressure of CO reached 1 atm, acetic acid production dropped to 20 mM and a small increase in the yield of methyl bisulfate was observed. The effect of CO partial pressure on the yield of methyl bisulfate is similar, but less dramatic than that on yield of acetic acid, for partial pressures below about 0.2 atm. Accordingly, in one embodiment of the invention of up to about 0.2 atm CO may be included in the feed to this process.

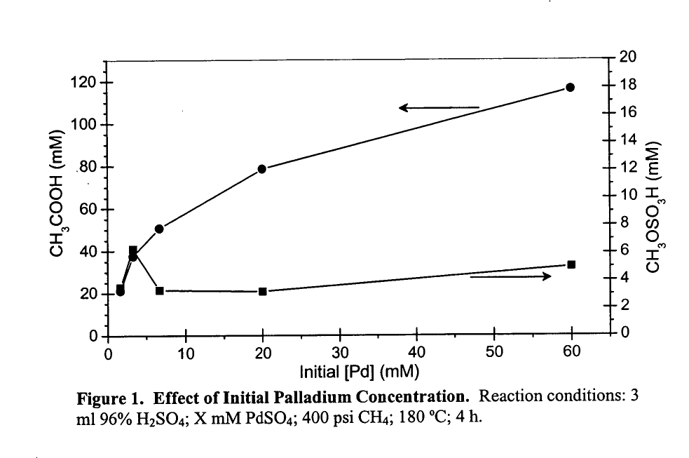

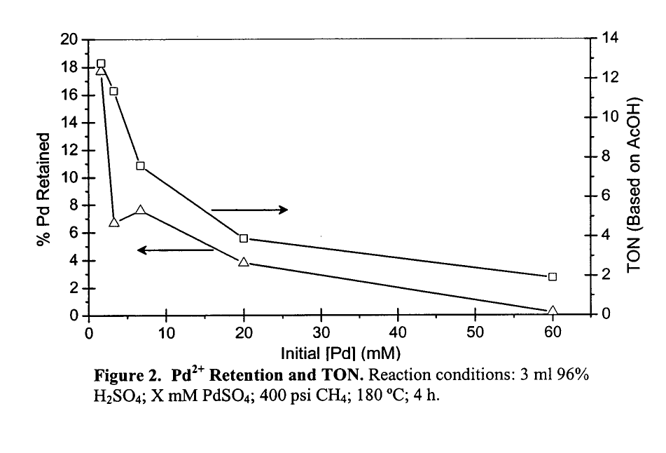

FIG. 1 illustrates the effects of the initial concentration of PdSO₄ on the yields of acetic acid and methyl bisulfate. The percentage of the initially charged PdSO₄ retained in solution at the end of the reaction, and the number of moles of acetic acid produced per mole of PdSO₄ charged to the reaction (TON) is shown in FIG. 2. The yield of acetic acid increases with the initial charge of PdSO₄ but with a decreasing slope, which leads to a decrease in the TON. By contrast, the yield of methyl bisulfate changes to a much lesser degree with increasing charge of PdSO₄. FIG. 2 shows that the percentage of Pd retained in solution at the end of reaction decreases as the magnitude of the initial charge of PdSO₄ increases. These latter results suggest that less than 1 mM of Pd²⁺ is retained in solution after reaction for the chosen reaction conditions, independent of the initial charge of PdSO₄. To test this hypothesis, a reaction was carried out starting with Pd-black. Acetic acid was formed (a yield of 37.9 mM after 4 h), and the final concentration of Pd²⁺ in solution was 0.45 mM. These results support the proposition that only a limited amount of Pd²⁺ can be maintained in solution during reaction. However, as demonstrated by the results presented in Tables 1 and 2, the percentage of Pd²⁺ retained in solution is a strong function of the ratio of O₂ to CH₄ partial pressures and, to a lesser extent, the total system pressure. Taken together, these results suggest that under reaction conditions the concentration of Pd²⁺ retained in solution is dictated by a balance between the rates of Pd²⁺ reduction and Pd⁰ oxidation.

The reaction temperature was found to affect the distribution of products and the retention of Pd²⁺ in solution. As shown in Table 3, decreasing the reaction temperature from 180° C. to 160° C. resulted in only a modest decrease in the yield of acetic acid. This was accompanied by a dramatic increase in selectivity, a noticeable increase in the yield of methyl bisulfate and Pd retention, and a significant decrease in the yields of methanesulfonic acid, methanedisulfonic acid, and sulfoacetic acid.

TABLE 3
Effect of Reaction Temperature. Reaction
conditions: 3 ml 96% H₂SO₄; 20 mM PdSO₄;
400 psi CH₄; 150 psi O₂; 4 h.

| | 180° C. | 160° C. |
|---|---|---|
| CH₃COOH (mM) | 284 | 266 |
| CH₃OH (mM) | 6.8 | 15.8 |
| CH₃SO₃H (mM) | 55.5 | 17.0 |
| HO₃SCH₂COOH (mM) | 16.3 | 4.3 |
| CH₂(SO₃H)₂ (mM) | 36.8 | 1.1 |
| S_{CH3COOH} (%) | 44 | 82 |
| S_{COx} (%) | 47 | 13 |
| Pd Retained (%) | 40 | 51 |

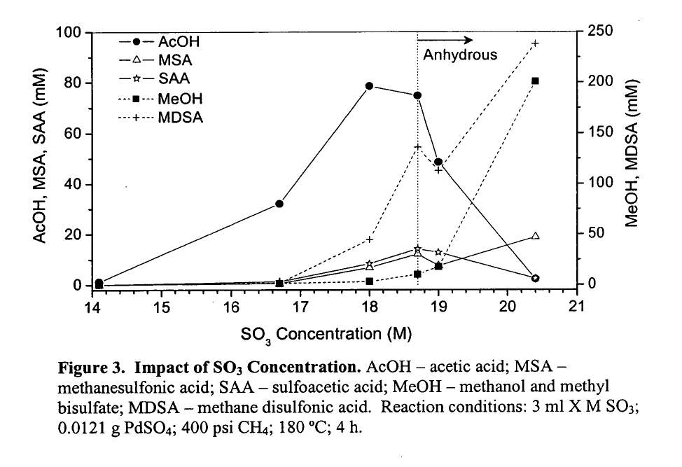

The effect of the strength of the sulfuric acid solution on the distribution of products was also investigated. FIG. 3 shows the yields of acetic acid, methyl bisulfate, methanesulfonic acid, methanedisulfonic acid, and sulfoacetic as a function of the concentration of SO₃. Below 18.7 M all of the SO₃ is present as H₂SO₄, and above this molarity an increasing fraction of the SO₃ is present as free SO₃ dissolved in 100% H₂SO₄. The yield of acetic acid increases to up to a maximum value of 78.5 mM at 18.0 M SO₃, where after it decreases to nearly zero. By contrast, the yield of methyl bisulfate is negligible for SO₃ molarities below 18.0, but rises rapidly thereafter. Below an SO₃ concentration of 18.0 mM the yields of methanesulfonic acid, methanedisulfonic acid, and sulfoacetic acid are very small. However, above this SO₃ concentration the yield of methanesulfonic acid increases from 6.7 up to 18.8 mM, and the yields of methanedisulfonic acid and methyl bisulfate increase sharply from 44.6 and 3.2 mM up to 238 and 201 mM, respectively, and the yield of sulfoacetic acid increases from 8.3 mM up to 14.1 mM.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference in their entirety for all purposes.

What is claimed is:

1. A process for the production of a product comprising acetic acid comprising contacting a methane-containing feed with an oxygen-containing gas in the presence of a palladium-containing catalyst and an acid selected from concentrated sulfuric acid and fuming sulfuric acid, wherein the oxygen partial pressure is from about 30 to about 300 psi, the methane partial pressure is from about 100 to about 1000 psi, and the total pressure of oxygen and methane is from about 130 to about 1300 psi, in the absence of a promoter for the catalyst.

2. A process according to claim 1, wherein the oxygen partial pressure is from about 60 to about 200 psi.

3. A process according to claim 1 wherein the methane partial pressure is from about 100 to about 400 psi.

4. A process according to claim 1, wherein the oxygen partial pressure is from about 60 to about 200 psi and the methane partial pressure is from about 100 to about 400 psi.

5. A process according to claim 1 wherein the total pressure of oxygen and methane is from about 200 to about 600 psi.

6. A process according to claim 1 wherein the molar ratio of oxygen pressure to methane is from about 1:20 to about 1:1.

7. A process according to claim 1 wherein the molar ratio of oxygen to methane is from about 1:4 to about 1:1.

8. A process according to claim 1 wherein the molar ratio of oxygen to methane is from about 1:3 to about 1:1.5.

9. A process according to claim 1 wherein the temperature is from about 100 to about 250° C.

10. A process according to claim 1 wherein the temperature is from about 150 to about 180° C.

11. A process according to claim 1 wherein the acid comprises from about 80 to about 130 wt. % sulfuric acid.

12. A process according to claim 1 wherein the acid comprises from about 95 to about 100 wt. % sulfuric acid.

13. A process according to claim 1 wherein the catalyst concentration is from about 0.001 to about 1.0 M.

14. A process according to claim 1 wherein the catalyst concentration is from about 0.001 to about 0.1M.

15. A process according to claim 1 wherein the catalyst is selected from the group consisting of palladium salts and ligand-modified palladium systems.

16. A process according to claim 1 wherein the catalyst comprises a palladium salt.

17. A process according to claim 1 wherein the catalyst comprises palladium (II) or palladium (IV) sulfate, chloride, nitrate, acetate, acetylacetonate, amine, or oxide.

18. A process according to claim 1 wherein the catalyst comprises a ligand-modified palladium system.

19. A process according to claim 18 wherein the ligand is selected from phosphines, nitrites, and amines.

20. A process according to claim 19 wherein the ligand is selected from triphenylphosphine, bis(diphenylphosphinopropane), bipyrimidine, bipyridine, acetonitrile, and benzonitrile.

21. A process according to claim 1 wherein the oxygen-containing gas is molecular oxygen.

22. A process according to claim 1 wherein the oxygen-containing gas is a commercial mixture of molecular oxygen with an inert gas and that contains predominantly oxygen.

23. A process according to claim 1 wherein the oxygen-containing gas is air or oxygen-enriched air.

24. A process according to claim 1 wherein the methane-containing feed is an essentially pure methane stream.

25. A process according to claim 1 wherein the methane feed is a methane stream that contains one or more impurities, a methane-containing stream that emanates from a chemical process unit, a natural gas stream, a methane-containing stream produced by a gas generator, a methane-containing off-gas, or a biogenic methane stream.

26. A process according to claim 1 wherein the methane feed further contains one or more of ethane, propane, butane and higher hydrocarbons.

27. A process according to claim 1 wherein the methane feed further contains one or more of methanol, dimethyl ether, methyl acetate and methyl bisulfate.

28. A process according to claim 1 wherein the product further comprises methyl acetate.

29. A process according to claim 1 wherein the product further comprises acetyl sulfate.
